(19) United States
(12) Patent Application Publication
(10) Pub. No.: US 2008/0103085 A1
(43) Pub. Date: May 1, 2008

(54) Compound

(75) Inventor: Fridtjof Schroder, Hettlingen (CH)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 11/722,146

(22) PCT Filed: Dec. 20, 2005
(86) PCT No.: PCT/CH05/00764

§ 371 Date: Aug. 27, 2007

(30) Foreign Application Priority Data
Dec. 25, 2004 (GB) 0428306.5

(51) Int. Cl. A61K 8/40; C07C 255/31; C11B 9/00

(52) U.S. Cl. 512/8; 558/434

(57) ABSTRACT

The compound 5-(2,2-dimethyl-cyclopropyl)-3-methyl-pent-2-enenitrile and its use in fragrance or flavour applications.

This invention relates to a novel compound.

The invention provides the compound 5-(2,2-dimethyl-cyciopropyl)-3-methyl-pent-2-enenitrile.

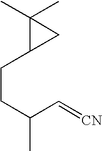

The compound may be made by any convenient method, an especially preferred method being the cyclopropanation of a suitable substituted alkene, comprising the reaction of the alkene with a carbenoide, generated from dibromomethane and a tri-(C₂-C₈)-alkylaluminum compound.

The compound is useful as an ingredient in fragrance or flavour applications. The invention therefore also provides the use in a fragrance or flavour application of 5-(2,2-dimethyl-cyclopropyl)-3-methyl-pent-2-enenitrile. The fragrance application may be in any field of fine or functional fragrance, for example, perfumes, toiletries, cosmetics, soaps, laundry and household products and polishes. The flavour application may be in any kind of food product or flavouring.

In such applications, it may be combined with other known fragrance or flavour materials to provide desirable fragrance or flavour properties.

The invention is further described with reference to the following example.

### EXAMPLE

Cyclopropanation of E/Z-Geranitrile: E/Z-5-(2,2-dimethyl-cyclopropyl)-3-nmethyl-pent-2-enenitrile.

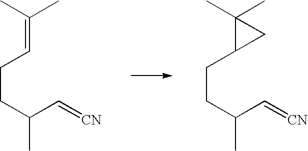

Anhydrous FeCl₃ (5 g, 30 mmol) is added under stirring and nitrogen to E/Z-Geranitrile (dr=1:1) (77 g, 0.52 mol) in dibromomethane (720 ml, 10.3 mol) at 10°-20° C., followed by slow addition of neat triisobutylaluminum (358 g, 1.8 mol) at this temperature. The mixture is stirred for 17 h at 25° C., then cooled to −10°-0° C. and pumped via double needle onto 25% NaOH at −10°-0° C. Under stirring the biphasic mixture is slowly warmed up to room temperature. The organic phase is separated, dried over MgSO₄, filtered and the solvent is evaporated under reduced pressure.

The thus obtained oily residue is subjected to the same cyclopropanation cycle as above using anhydrous FeCl₃ (5 g, 30 mmol), dibromomethane (720 ml, 10.3 mol) and neat triisobutylaluminum (358 g, 1.8 mol). After 17 h at 25° C. the mixture is cooled to −10°-0° C. and pumped via double needle onto 25% NaOH at −10°-0° C. Under stirring the biphasic mixture is slowly warmed up to room temperature. The organic phase is separated, washed with 4% oxalic acid, then with conc. NaHCO₃ until pH˜8, dried over MgSO₄ and filtered. After evaporation of the solvents under reduced pressure the oily residue is purified by distillation (bp 95° C. /0.06 Torr) giving 50 g (59%) of the cyclopropanation product (71% GC-purity, 8% aldelhyde, 11% alcohol, dr=1:1) as colorless oil. ¹H-NMR (CDCl₃, 400 MHz): 5.1 (1 H), 2.5 (m, 1 H), 2.25 (m, 1 H), 2.05 (s, 1.5 H), 1.9 (s, 1.5 H), 1.6- 1.3 (2 H), 1.05 (6 H), 0.45 (2 H), −0.1 (1 H) ppm. ¹³C-NMR (CDCl₃, 400 MHz): 165.43 and 165.4 (2 s), 117.2 and 117.0 (2 s), 95.6 and 95.0 (2 d), 39.1 and 36.7 (2 t), 27.6 and 27.5 (2 t), 27.39 and 27.37 (2 q), 28.9 and 23.8.(2 d), 22.9 and 21.0 (2 q), 19.9 and 19.8 (2 q), 15.6 and15.5 (2 s) ppm, GC/MS: 162 (20%), 148 (1%, [M-15]⁺), 94 (20%), 81 (55%), 55 (100%). IR (film): 2952 (s), 2867 (m), 2218 (w), 1676 (w), 1632 (w), 1454 (m), 1377 (m), 1365 (m), 1120 (w), 1020 (m), 866 (w), 801 (w). Odour: Hesperidic, powerful, fresh, geranitrile.

1. 5-(2,2-dimethyl-cyclopropyl)-3-methyl-pent-2-enenitrile.

2. Use of 5-(2,2-dimethyl-cyclopropyl)-3-methyl-pent-2-enenitrile in a fragrance application.

3. A fragrance application comprising 5-(2,2-Dimethyl-cyclopropyl)-3-methyl-pent-2-enenitrile.

4. Use of 5-(2,2-Dimethyl-cyclopropyl)-3-methyl-pent-2-enenitrile in a flavour application.

5. A flavour application comprising 5-(2,2-dimethyl-cyclopropyl)-3-methyl-pent-2-enenitrile.

6. The use of 5-(2,2-dimethyl-cyclopropyl)-3-methyl-pent-2-enenitrile in a fragrance application according to claim 2 wherein the fragrance application is a fine or functional fragrance.

7. The use of 5-(2,2-dimethyl-cyclopropyl)-3-methyl-pent-2-enenitrile in a fragrance application according to claim 2 wherein the fragrance application is selected from: perfumes, toiletries, cosmetics, soaps, laundry and household products, and polishes.

8. Use of 5-(2,2-Dimethyl-cyclopropyl)-3-methyl-pent-2-enenitrile according to claim 4 wherein the flavour application selected from food products or flavorings.
